(19) United States
(12) Patent Application Publication
(10) Pub. No.: US 2020/0061210 A1
(43) Pub. Date: Feb. 27, 2020

(54) NOVEL METHOD FOR GENE THERAPY USING INTRANASAL ADMINISTRATION OF GENETICALLY MODIFIED VIRAL VECTORS

(71) Applicant: BioViva USA, Inc., Bainbridge, WA (US)

(72) Inventors: Elizabeth Louise Parrish, Bainbridge Island, WA (US); Anca Selariu, FORT COLLINS, CO (US)

(21) Appl. No.: 16/272,956

(22) Filed: Feb. 11, 2019

Related U.S. Application Data
(60) Provisional application No. 62/723,469, filed on Aug. 27, 2018.

(51) Int. Cl. A61K 48/00

(52) U.S. Cl. CPC A61K 48/0075; A61K 48/0091

(57) ABSTRACT

A novel method for gene therapy using intranasal administration of genetically modified viral vectors. Target genes for therapeutic administration to a human patient, for many purposes such as production of telomerase in a patient's body, are selected and transfected into a bacterial cell. Viral transduction is used to infect a human-administrable virus with the target genes in the bacterial cell. A solution containing a therapeutic amount of transfected viral agents is intranasally administered the solution into a human patient.

### CROSS-REFERENCE TO RELATED APPLICATIONS

This application is a continuation-in-part of U.S. provisional patent application Ser. No. 62/723,469, titled “NOVEL METHOD FOR GENE THERAPY USING INTRANASAL TRANSFECTION OF HCMV” and filed on Aug. 27, 2018, the specification of which are incorporated herein by reference in their entirety

### BACKGROUND OF THE INVENTION

### Field of the Art

The disclosure relates to the field of gene therapy, specifically to the field of low-cost designable gene therapy treatments.

### Discussion of the State of the Art

It is currently the case that gene therapy for patients is exceedingly expensive and limited in implementation, allowing only a small number of genes or only a single gene to be inserted into a patient through a single delivery method. It is further the case that there is no commercially viable method to extend the telomeres at the ends of chromosomes in human cells, in an effort to extend lifespans through the use of telomerase. If a patient requires multiple genes or pieces of genetic code inserted for medicinal purposes, for example to extend telomeres at the end of a cell and to also protect against certain genetic disorders (or even remove them entirely, should such genetic structuring become available in the future), they may have to have gene therapies administered multiple times or in multiple different ways, raising the cost even further than what it already is, and many gene therapy techniques may not quickly or effectively achieve full saturation in a patient's body. A solution that allows for multi-gene cassettes to be inserted easily into a patient must be devised, which may allow for multi-genetic treatments, important for many health issue and potential life-changing effects. It is known that so-called monogenetic traits are rare in most complex organisms, compared to traits and issues caused by numerous genetic factors and external factors, and while external factors may be accounted for and dealt with adequately in many cases by modern medical practice, dealing with genetic factors, much less numerous genetic factors such as multiple gene mutations working to cause numerous issues in a patient, still pose problems both with efficacy and cost to patients. The lifespan of an individual could theoretically reach as much as 120 years with appropriate, effective, and affordable gene therapy techniques, as well as countering many genetic disorders and diseases or even curing some incurable disorders or diseases in the human species. A method to effectively, long-lastingly, and affordably insert a plurality of genetic code sequences into a host body for therapeutic purposes would potentially achieve this. A human cytomegalovirus (“HCMV”) or varicella zoster virus (“VZV”) would achieve these goals of viable multi-gene insertion, and be reactivatable in a host body, while the virus itself is asymptomatic in non-immunocompromised individuals, making it an optimal delivery method for affordable, effective gene therapy. As well, telomerase reverse transcriptase (“TERT” or “ten”) encoding genes may be used to produce a key enzyme in the reversal of telomere degradation, the degradation of telomeres in humans being linked to shorter lifespans, cancers, and certain genetic disorders such as cri du chat, a disorder which causes numerous problems for children affected, and is caused by partial deletion of the short arm of chromosome 5. The absence of human TERT or hTERT is heavily associated with this disorder, and may be preventable with the disclosed techniques.

What is needed is a human cytomegalovirus and method for gene insertion into such a virus for the purposes of gene therapy.

### SUMMARY OF THE INVENTION

Accordingly, the inventor has conceived and reduced to practice, in a preferred embodiment of the invention, a novel method for gene therapy using intranasal administration of genetically modified viral vectors such as HCMV or VZV. The following non-limiting summary of the invention is provided for clarity, and should be construed consistently with embodiments described in the detailed description below.

To solve the problem of a lack of a cheap and effective system for multi-gene insertion into target host cells for gene therapy, a novel method for gene therapy using intranasal administration of genetically modified viral vectors has been devised, utilizing techniques for viral transfection (also known as viral transduction) of cells, polymerase chain reactions (PCR) to grow desired genetic components, utilizing a possible cytomegalovirus for transcription of many different genetic coding sequences into a host body, and intranasal administering for easy, cheap, and effective metabolizing into the entire host body through the liver. Target genes may be selected for a given patient's therapy, prepared and bred through bacterial artificial chromosome (BAC) growth and PCR techniques, viral transduction, and then a solution may be prepared containing viruses with the desired genes, before being administered into a patient intranasally.

The inventor has conceived and reduced to practice a novel method for gene therapy using intranasal administration of genetically modified viral vectors, comprising: selecting target genes for a patient's gene therapy; using polymerase chain reactions to create at least one desired gene with recombination sites; wherein a desired gene may be extracted from a bacterial, animal, or plant cell, or virus; creating an empty coding sequence consisting of identical recombination sites to those of the desired genes; wherein an empty coding sequence also contains a leading and trailing untranslated region; recombining a desired target gene into an empty coding sequence, thereby creating a full genetic coding sequence capable of creating proteins in a cell; wherein recombination of a desired target gene takes place in a bacterial cell; sequencing bacterial DNA to ensure that the desired gene is present in the bacterial DNA; performing viral transduction to infect a desired virus with the sequenced target gene; producing a solution containing a sufficient amount of viral agents to introduce to a patient effectively; and administering a solution containing viruses with the desired genetic traits for infection of a human host intranasally.

### BRIEF DESCRIPTION OF THE DRAWING FIGURES

The accompanying drawings illustrate several aspects and, together with the description, serve to explain the principles of the invention according to the aspects. It will be appreciated by one skilled in the art that the particular arrangements illustrated in the drawings are merely exemplary, and are not to be considered as limiting of the scope of the invention or the claims herein in any way.

FIG. 1 is a method diagram of high-level steps needed for intranasal multi-gene HCMV or VZV insertion into a patient's body, according to a preferred embodiment.

FIG. 2 is a method diagram of high level steps taken for generation and experimental verification of desired genes in an HCMV or VZV virus, according to a preferred embodiment.

FIG. 3 (PRIOR ART) is a diagram of an HCMV cell and important components of such.

FIG. 4 is a diagram of the lifecycle of a patient's cells once infected with HCMV or VZV.

FIG. 5 is a diagram of the construction of a gene expression cassette for implementation in a HCMV or VZV for delivery into a patient.

FIG. 6 is a diagram of a partial process of double homologous recombination of genes, according to a preferred aspect.

FIG. 7 is a diagram of a partial process of double homologous recombination of genes, according to a preferred aspect.

FIG. 8 is a method diagram of gene recombination and construction of desired genes for insertion into a HCMV or VZV.

FIG. 9 is a diagram of the construction of a gene expression with a plurality of genes inserted into a single cassette, for application in a HCMV or VZV, according to a preferred aspect.

FIG. 10 is a method diagram of HCMV or VZV delivery intranasally and the process of being metabolized into the host body through the liver, according to a preferred embodiment.

FIG. 11 (PRIOR ART) is a diagram of key steps in a method for bacterial gene recombination to acquire desired genes for later viral transfection.

FIG. 12 (PRIOR ART) is a diagram of key steps in a method for bacterial gene recombination to acquire desired genes for later viral transfection.

FIG. 13 is a method diagram showing steps in the creation of mouse cytomegalovirus (MCMV)-Luc SW102 electro competent cells, as an alternative method of introducing desired genes into a cytomegalovirus for administration, according to a preferred aspect.

FIG. 14 is a diagram showing the insertion of an m-tert gene at a specific locus in a bacterial artificial chromosome in a mouse cytomegalovirus (MCMV).

### DETAILED DESCRIPTION

The inventor has conceived, and reduced to practice, a novel method for gene therapy using intranasal administration of genetically modified viral vectors.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

### Conceptual Architecture

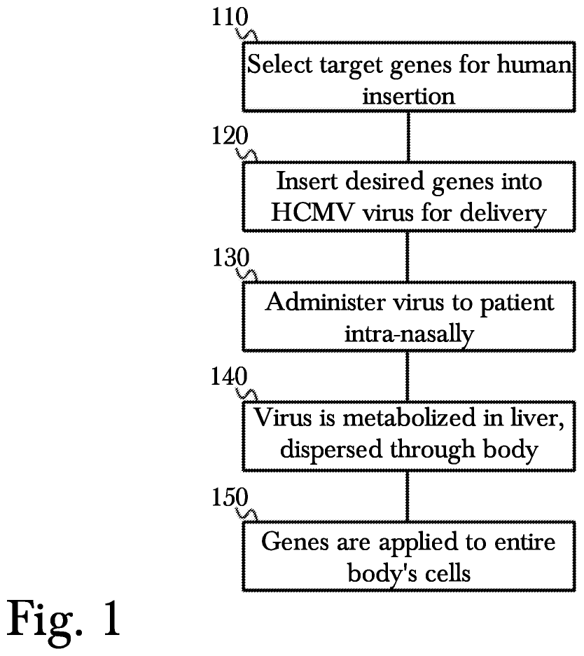

FIG. 1 is a method diagram of high-level steps needed for intranasal multi-gene HCMV or VZV administration into a patient's body, according to a preferred embodiment. First, target genes are selected for human insertion 110 according to a desired outcome or change in patient genetics, which may fluctuate and alter greatly depending on a patient's individual medical needs and their own genetic makeup. Such genes may include a TERT gene for insertion to promote telomerase reverse transcriptase enzyme production, in an effort to lengthen telomeres in a target patient, and other genes, as needed. Genes selected may be a plurality of genes or only one target gene, as required for a patient's therapy. Such genes, once selected, are inserted into the HCMV or VZV virus 120, through processes elaborated upon further in this application, including bacterial recombination and viral transfection. A solution containing the virus is inserted into one of several possible intranasal administering devices, including a dropper or sprayer, and then administered to the patient 130 intranasally, which allows for fast absorption into the body, where it may then be metabolized by the liver and dispersed through the body 140. Once this occurs, based on the lifecycle of infected cells and the HCMV or VZV virus in the host body, the majority of the body's cells are then infected with whatever gene cassettes may be carried by the HCMV or VZV 150, thus allowing for fast and effective gene therapy, provided an HCMV or VZV virus may be programmed with selected genes 120.

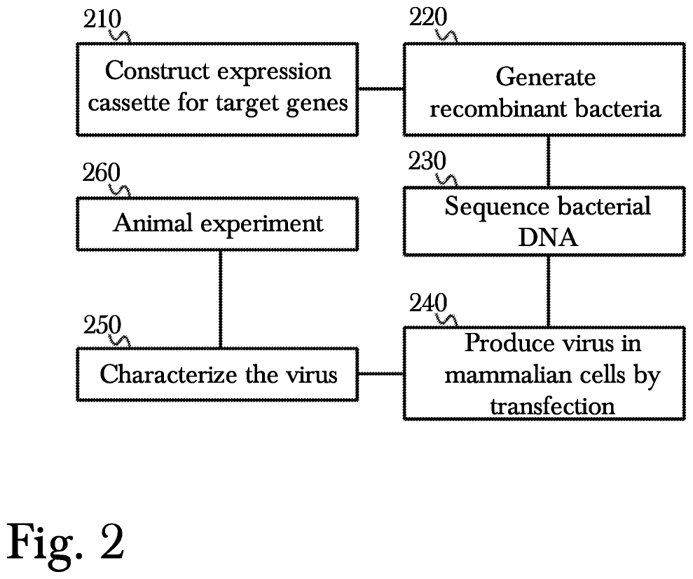

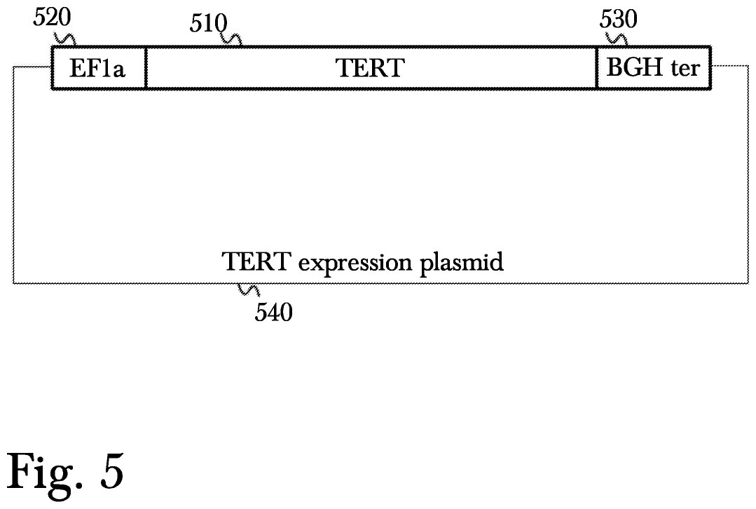

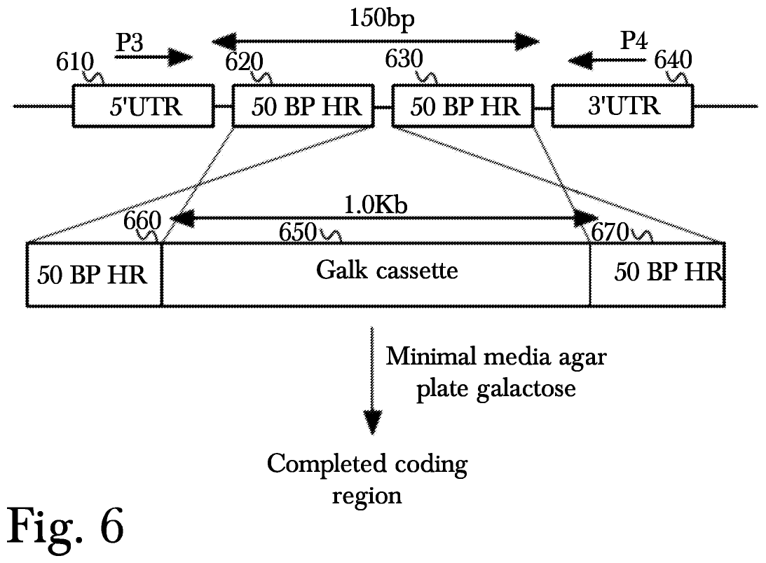

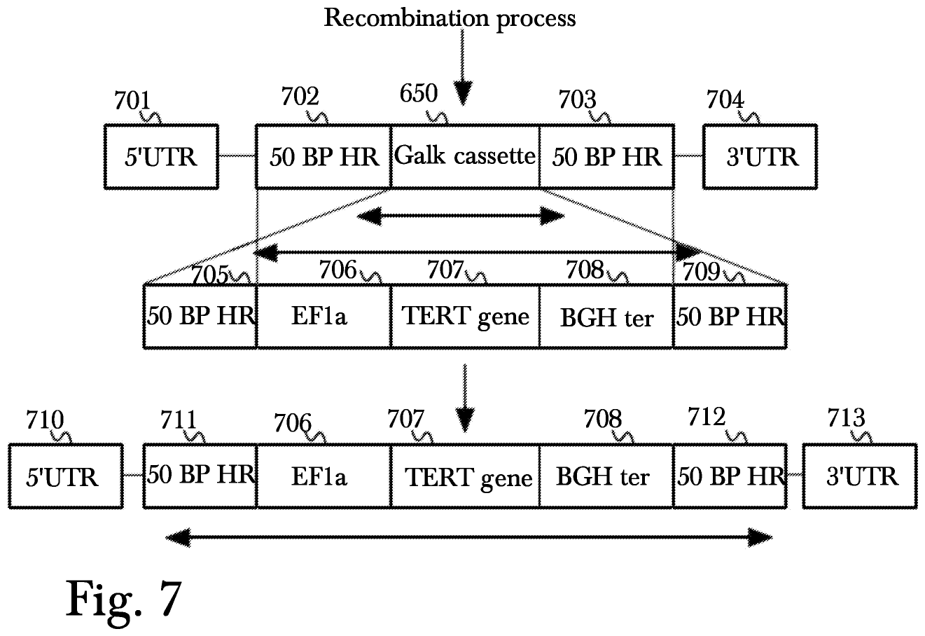

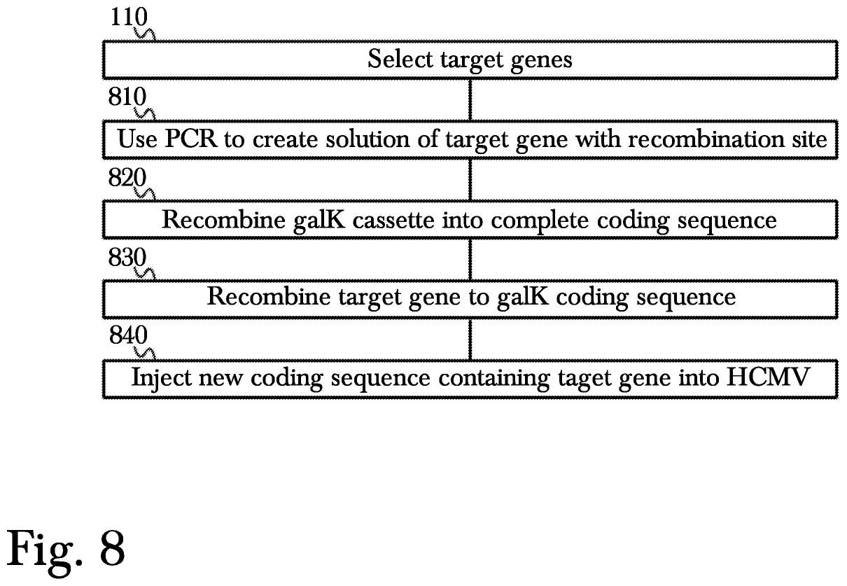

FIG. 2 is a method diagram of high level steps taken for generation and experimental verification of desired genes in an HCMV or VZV virus, according to a preferred embodiment. First, constructing an expression cassette for target genes 210 must be accomplished FIG. 5, FIG. 6, FIG. 7, FIG. 8. A gene cassette may contain one or a plurality of genes, including galK gene, or hTERT or mTERT genes, which may code for multiple enzymes and proteins, depending on a patient's treatment plan. In particular, telomerase reverse transcriptase genes may allow for the patient to produce a key enzyme in the production and maintenance of telomeres at the end of chromosomes within their cells, which may prolong lifespan and produce other benefits in a patient. Recombinant bacteria is generated 220, the purpose of which is to create specific proteins and RNA, important steps in the creation of gene cassettes for transfection of viral cells for later administering to a human. When desired bacteria are created with the desired genes and gene expressions, its DNA is sequenced 230, and recombinant BAC's (Bacterial Artificial Chromosome) are transfected to mammalian cells such that recombinant viruses may be produced 240. BACs are plasmids that represent constructed DNA segments which can represent specific desired genes, and can be grown, sequenced out of a bacterium, or transferred to other cells, depending on the utilization desired by the practitioner or practitioners involved. Once a virus is produced 240, the virus is characterized 250 to ensure it has the correct genetic cassettes for insertion, before it is tested on animals 260 to ensure viability and efficacy. Such a virus operates through viral transduction, a type of transfection whereby a virus may be the carrier for genetic material into host cells.

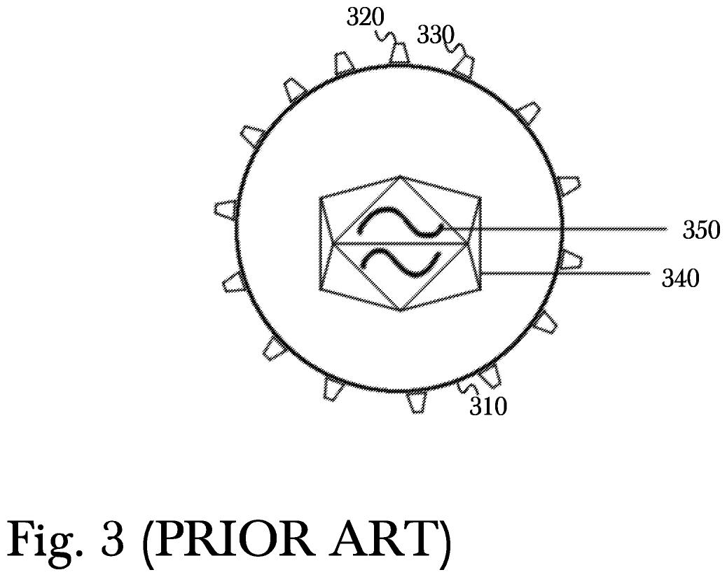

FIG. 3 (PRIOR ART) is a diagram of an HCMV cell and important components of such. A viral envelope 310 exists, which may be derived partially from phospholipids and proteins common in human cells, but also contains glycoproteins 320, 330. A viral envelope 310 encapsulates a capsid 340, a protein shell which protects and contains the genetic material 350 of the virus. Such genetic material may include a TERT gene or other genes as desired by a healthcare practitioner for administering to a patient, for the purpose of treating disorders caused by lacking the hTERT enzyme, rebuilding and growing the telomeres in patient cells, and more. A viral envelope 310, containing the viral capsid 340, may consist of alternating glycoproteins 320, 330 for binding to receptor sites on host cells. Inside a capsid 340 is the genome of the virus 350, containing the virus' genetic code, ideally containing gene cassettes built from selected genes 110 that are to be administered to a patient.

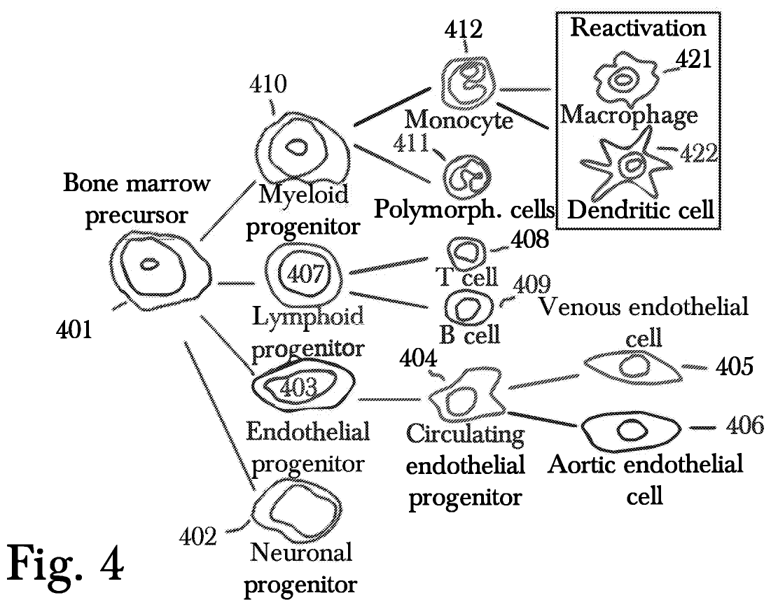

FIG. 4 is a diagram of the lifecycle of a patient's cells once infected with HCMV or VZV. A bone marrow precursor cell 401, sometimes referred to as a sort of unipotent cell which is a stem cell that has differentiated slightly and lost many of its stem cell properties. However, bone marrow precursors can become many different types of cells in the body, and once infected with a HCMV or VZV virus payload containing desired genetic coding sequences, may deliver these sequences to the rest of the body effectively. A bone marrow precursor cell 401 has several lineages of transformation it may undergo, one of which being that to a neuronal progenitor 402, which has the ability to further differentiate into a finite number of neuronal and glial cell types later on. Another lineage of transformation or differentiation that a bone marrow precursor cell 401 may undergo includes an endothelial progenitor 403, the endothelial lining being a key component in blood vessel biology inside the human body and many other animal bodies. Such a progenitor cell 403 may further differentiate into a circulating endothelial progenitor cell 404, and then possibly a venous endothelial cell 405 or an aortic endothelial cell 406, in this way achieving viral spread throughout the cardiovascular system. A separate lineage of differentiation for further bodily infection that is possible once bone marrow precursor cells 401 are infected is that of a lymphoid progenitor 407. The lymphoid progenitor 407 can differentiate into types of cells such as T-cells 408 and B-cells 409, key components in the body's immune system. Another lineage of differentiation that is possible for a bone marrow precursor 401 is a myeloid progenitor 410, which may further differentiate into polymorphic cells 411 and monocytes 412, which are a form of white blood cell and capable of differentiating into a macrophage 421 or a dendritic cell 422, both of which are important concepts in the reactivation of the HCMV or VZV virus in the body.

FIG. 5 is a diagram of the construction of a gene expression cassette for implementation in a HCMV or VZV for delivery into a patient. Shown here is an example of an expression plasmid which may be utilized, any number of different genes may be selected for an expression plasmid rather than a specific TERT gene, for varying treatment plans. A plurality of gene expressions may also be present, rather than a single one, as will be shown in later drawings. Shown is a TERT expression plasmid 540 also known as an expression vector. The main components of such an expression vector are the gene to be transferred into target cells 510, in this example a TERT gene being chosen for patients who may require, for example, increased production of telomerase reverse transcriptase, an important enzyme in the creation of telomerase and the extension of chromosomal telomeres. Extending telomeres of patient cells is a new concept which may alleviate or even be able to prevent certain disorders such as cri du chat, a genetic disorder characterized by a patient's inability to synthesize hTERT due to a chromosomal mutation, and may prolong lifespan or alter a patient's risk for various forms of cancer. A promoter piece of DNA 520 shown here as an EF1a promoter, whose purpose is to begin transcription of the target gene in the host cell. Also present is a BGH terminator 530 whose purpose is to terminate the sequence upon transcription, thus providing us with a TERT cassette in a usable expression plasmid 540. This is a mono-gene cassette, with only one gene inside the plasmid, however it should be obvious to those skilled in the art that multiple genes may be used a multi-gene cassette, and thereby multiple proteins and enzymes may be coded for in the genetic material inserted into a patient, for many potential treatments.

FIG. 6 is a diagram of a partial process of double homologous recombination of genes, according to a preferred aspect. A plurality of 50 base-pair homologous recombination sites 620, 630 exist in a HCMV or VZV bacterial artificial chromosome (BAC), between a 5′ untranslated region 610 and a 3′ untranslated region 640 which mark directional endpoints of an incomplete cassette. Using homologous recombination sites 620, 630, a galK gene cassette 650, which exists as a piece of genetic code between two similar or identical 50 base-pair homologous recombination sites 660, 670, is then recombined and translated into the initial structure containing recombination sites 620, 630 and directional endpoints 610, 640, resulting in a structure as shown in FIG. 7.

FIG. 7 is a diagram of a partial process of double homologous recombination of genes, according to a preferred aspect. A galK gene cassette 650 is recombined as shown in FIG. 6 and may exist in a HCMV or VZV BAC and now has two 50 base-pair homologous recombination sites 702, 703, and a 5′ UTR 701 leader sequence and 3′ UTR 704 trailer sequence, making the entire structure a completed coding sequence capable of correctly encoding for desired proteins—in this instance, those coded for by a galK gene, and capable of being injected via a HCMV or VZV or similar virus. A second instance of homologous recombination then takes place with a gene cassette for a TERT gene 707, including an EF1a promoter 706 and BGH terminator 708, and two 50 base-pair recombination sites 705, 709. Recombination with the previous coding sequence results in a completed coding sequence for a TERT gene 707 encapsulated by an EF1a promoter 706, BGH terminator 708, two 50 base-pair recombination sites 711, 712 and a leading 5′ untranslated region 710 and a trailing 3′ untranslated region 713, ready to administer to a patient via a carrier HCMV or VZV. This general process of multi-step homologous recombination using gene cassettes and BAC's results in a HCMV or VZV BAC, or in other words, a piece of genetic code compatible and ready for insertion into an HCMV or VZV, to be administered to a patient. Through the process detailed in FIG. 6 and FIG. 7, various genes may be recombined and inserted into cassettes able to be inserted into bacteria and transfected into other organisms, and it will be obvious to those with ordinary skill in the art that the process of double homologous recombination that numerous other genes may be transcribed here for numerous differing treatments, which may range from treating genetic disorders previously thought to be untreatable, to promoting increased protein production for other treatments, and possibly even cancer treatments for different forms of cancerous tumors by encoding genes which terminate the cancerous cells, depending on what genes are encoded for therapy.

FIG. 8 is a method diagram of gene recombination and construction of desired genes for insertion into a HCMV or VZV. Target genes are selected 110 for human insertion, the selected genes varying depending on specific medical requirements for a given patient. Gene selection may be based on prolonging human life, as with selecting the hTERT gene to eventually be administered to a human patient, or may be based on attempting to cure a genetic disorder, for example cri du chat, Down syndrome, and others, possibly including cancer-causing mutations. Polymerase chain reactions (PCR) are used to create many copies of a desired gene with recombination sites 810 in a solution, as is common in the art, using thermocycling as a key part of the PCR technique for DNA melting and enzyme action to take place. Recombination of a galK cassette 650 into a complete coding sequence 820 then may take place, using an empty coding sequence which contains homologous recombination sites 620, 630 to write a galK cassette into a coding sequence 820 before a galK cassette is recombined into a target gene 830. An example of a target gene that can be recombined into a coding sequence may be a TERT gene 707, for the purpose of aiding a patient in production of telomerase and extending telomeres at the ends of patient cells, for example. Once a new coding sequence is completed it may be injected into a HCMV or VZV 840, FIG. 3, for later administering to a patient intranasally 130, 1020.

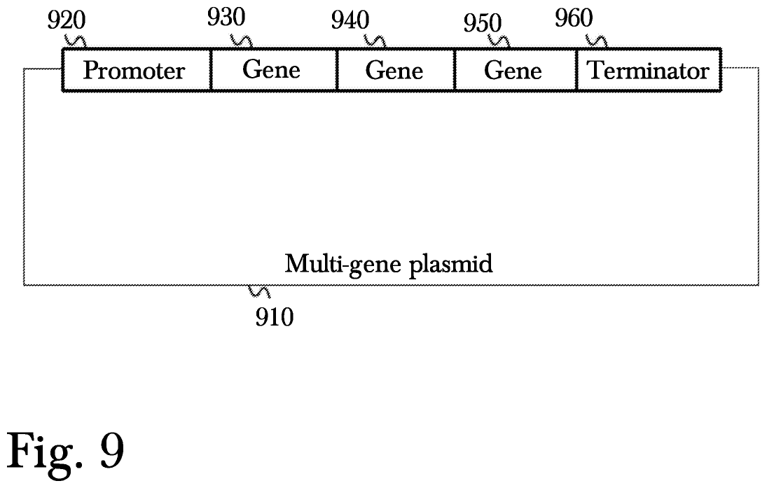

FIG. 9 is a diagram of the construction of a gene expression with a plurality of genes inserted into a single cassette, for application in a HCMV or VZV, according to a preferred aspect. FIG. 9 shows a similar diagram to that of FIG. 5 but rather than having a single gene plasmid, a possible three-gene plasmid 910 is shown, to illustrate the possibility of a multi-gene plasmid. A promoter 920 exists at one end of the plasmid's genetic code, existing to encourage and start the transcription of the rest of the genetic code of the plasmic, contained in multiple genes 930, 940, 950. A termination string of genetic code 960 exists to stop transcription and signals the end of the genetic code contained in the plasmid. It is possible to have multiple genes, not simply three or one gene, inside a plasmid, which is what this diagram conveys here. It would be possible to one with ordinary skill in the art to be able to understand the synthesis of a plasmid with more than one gene, provided a suitable delivery method exists, for which the HCMV or VZV is ideal.

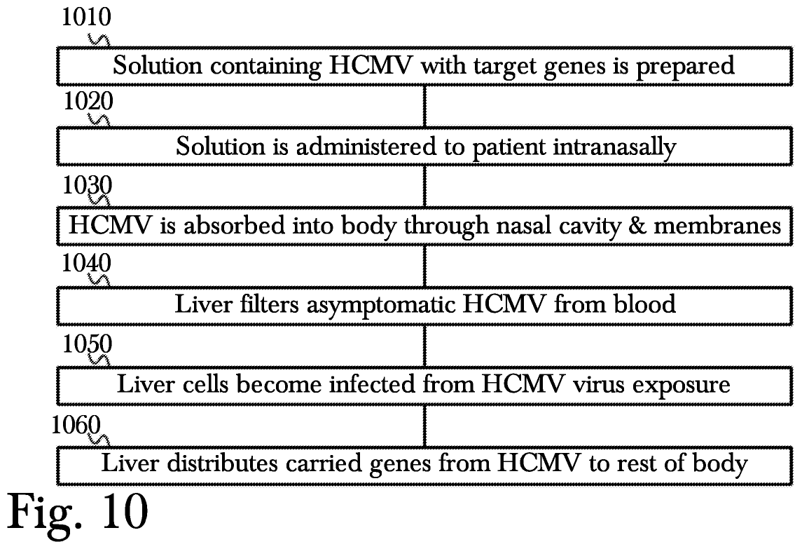

FIG. 10 is a method diagram of HCMV or VZV delivery intranasally and the process of being metabolized into the host body through the liver, according to a preferred embodiment. First, a solution containing HCMV or VZV with desired genetic material must be prepared 1010. This may be accomplished through the processes described earlier including using homologous recombination sites to produce gene cassettes containing desired genetic material as in FIG. 6, FIG. 7, insertion of gene cassettes into viral agents and administering to a patient, or some other method. Target genes may be selected from a pool of potential candidate genes, which may allow for treatment of many different disorders or ailments for a patient, or improve their health even in the absence of a disorder, such as with selecting the hTERT gene to extend patient telomeres and potentially prolong lifespans. A solution, once prepared 1010, may then be introduced or administered to a patient 1020, including intranasal administration. Intranasal administration of therapeutic solutions provides a cheap and effective way to introduce a gene into an individual's body, allowing it to be absorbed through the nasal cavity and membranes present in the nasal passages of a patient 1030. Once absorbed this way, the viral agent containing the desired gene or genes may then be processed by a patient's liver 1040, which infects liver cells 1050 with the genetic material, and can result in efficient propagation of selected genes through a patient's body 1060, through the bloodstream. In this way, genes may be selected 1010, grown through numerous techniques outlined herein, transfected into a viral agent, administered to a patient 1020, and spread through the patient's body rapidly 1060, resulting in cheap, effective gene therapy for a patient.

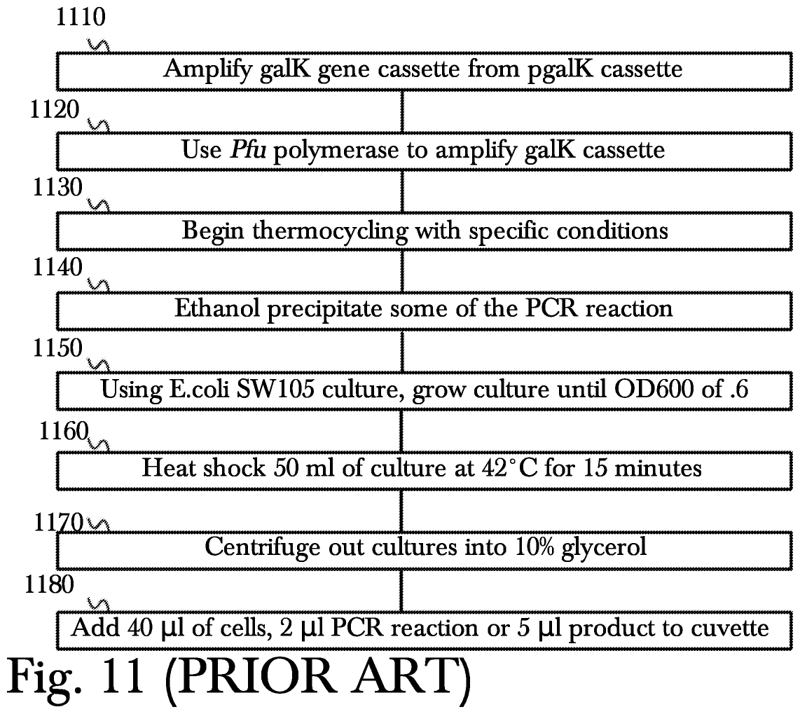

FIG. 11 (PRIOR ART) is a diagram of key steps in a method for bacterial gene recombination to acquire desired genes for later viral transfection. First, a galK gene cassette must be amplified from a pgalK template 1110 using specific primers comprising the nucleotide sequence of 5′-CCTGTTGACAATTAATCATCCGCA-3′, or a reverse primer consisting of 5′-TCAGCACTGTCCTGCTCCTT-3′. Using a polymerase chain reaction (PCR) technique, involving the use of a pfu DNA polymerase enzyme 1120, one may essentially grow many more copies of desired genetic code segments. PCR requires the use of thermocycling 1130 with specific laboratory conditions, which allows for DNA melting and enzyme action to take place, essentially allowing many complex reactions to take place over and over as temperatures are raised and lowered with specific regularity, as is common in the art. Ethanol is used to precipitate a portion of the PCR reactions out 1140, for further use, and for washing. Using E. coli SW105 culture, one may then grow a culture of E. coli until OD600 of 0.6 is achieved 1150, indicating a specific desired concentration, before heat shocking 50 ml of the culture at 42° C. for 15 minutes 1160 which induces expression of λRed recombinase genes. One may then centrifuge out cultures into 10% glycerol in two tubes 1170, possibly more than one time and washing in glycerol each time, to achieve a concentration of approximately 200-250 microliters of cells in each tube in 10% glycerol. Electroporation can then be started, firstly by adding 40 μl of cells, 2 μl of the PCR reaction or 5 μl of the ethanol-precipitated PCR product, to a cuvette 1180.

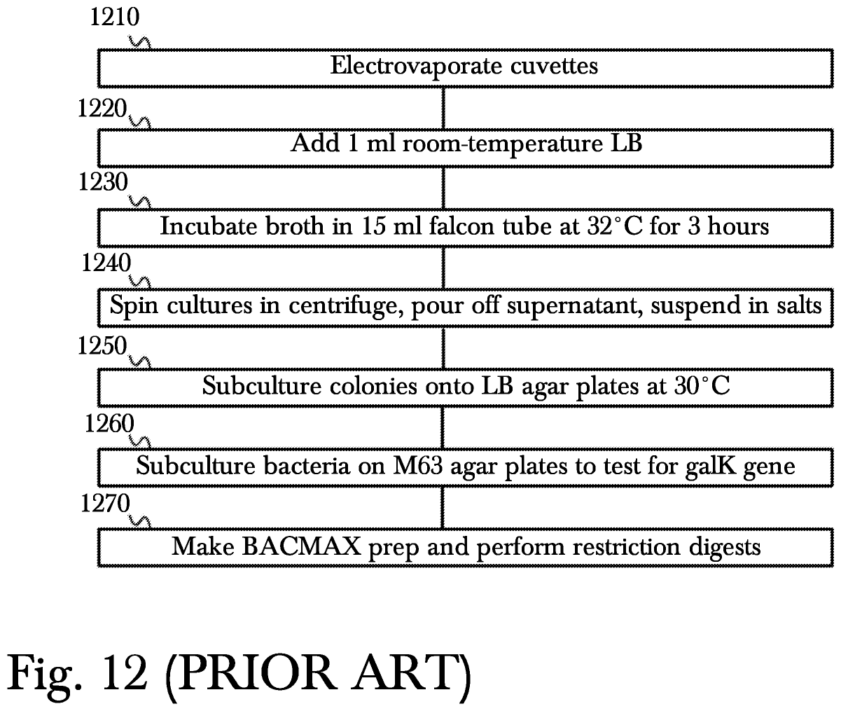

FIG. 12 (PRIOR ART) is a diagram of key steps in a method for bacterial gene recombination to acquire desired genes for later viral transfection. As shown in FIG. 11, once requisite materials and cell are added to a cuvette 1180, electroporation may be conducted 1210, before adding 1 ml of room-temperature lysogeny broth (LB) 1220. This allows cultures to grow in a nutrient rich environment. Incubation of broth can then be performed, in a 15 ml falcon tube at 32° C. for 3 hours 1230, with shaking, so as to mix the broth around. After incubation, cultures are spun at high speed for 5 minutes before supernatant is poured off and the cultures are re-suspended in a solution of 1×M9 salts 1240 comprising 48 mM Na2HPO4, 22 mM KH2PO4, 8.5 mM NaCl, and 18.6 mM NH4Cl. Cultures are then subcultured onto fresh LB agar plates at 30° C. and left to grow overnight 1250, and when they have grown overnight, test that the galK gene makes the cultures sensitive to 2-deoxy-galactose by subculturing them further onto M63 agar plates 1260. Lastly in this process, a lack of unwanted recombination of the BAC in repeat regions is confirmed by making a BACMAX prep and performing multiple restriction digests 1270.

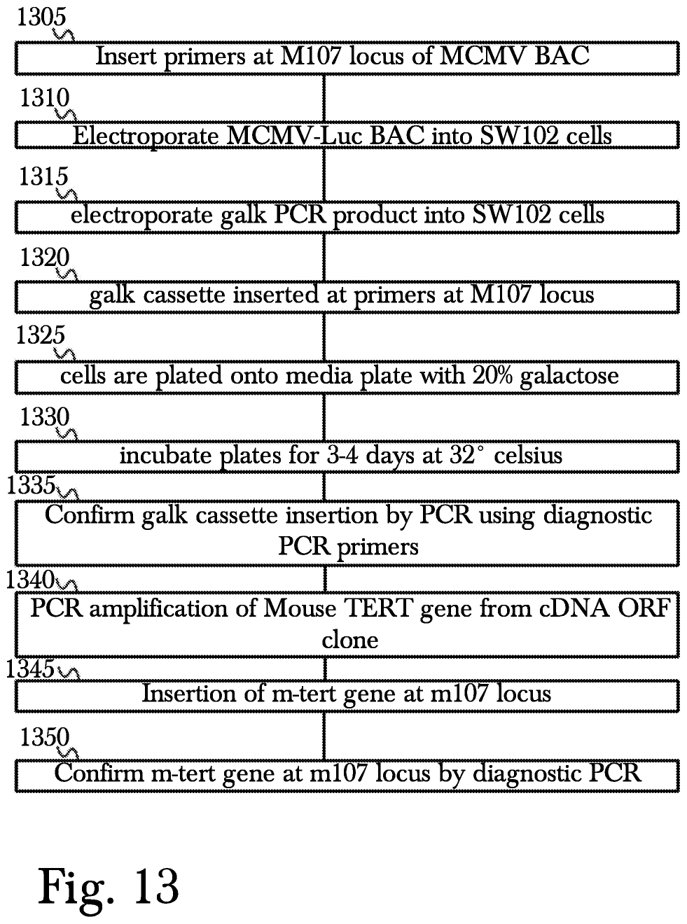

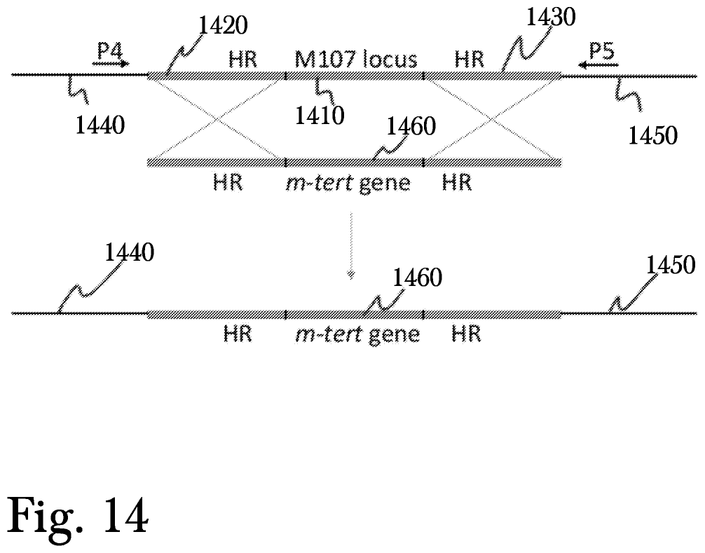

FIG. 13 is a method diagram showing steps in the creation of mouse cytomegalovirus (MCMV)-Luc SW102 electro competent cells, as an alternative method of introducing desired genes into a cytomegalovirus for administration, according to a preferred aspect. Primers are inserted via polymerase chain reactions, at the M107 locus of an MCMV BAC 1305, before the BAC is electroporated into SW102 cells 1310. galK polymerase chain reaction (PCR) products are then also electroporated into the cells 1315, where a galK cassette is inserted at the location of the primers in the M107 locus 1320. Mouse cytomegalovirus cells may be used in this methodology as an alternative to human cytomegalovirus (HCMV) cells. The cells may be placed on a media plate with a 20% galactose solution 1325 for growth of the cells, and incubated at 32 degrees Celsius for three to four days 1330 for optimal growth time. Confirmation of galK cassette insertion by PCR using diagnostic PCR primers 1440, 1450, 1335, to ensure successful recombination. The m-tert gene may be amplified by PCR techniques from a complimentary DNA (cDNA) open reading frame (ORF) clone 1340, which means to create clones of a plasmid containing cDNA for an m-tert gene using PCR. The gene will be recombined at the m107 locus in the MCMV BAC 1345, as shown in FIG. 14, at which point diagnostic primers 1440, 1450 may be used again to confirm the presence of desired genes 1350. Creation of plasmids in this manner may be useful for transfection into other cells including human cytomegaloviruses (HCMV), among other uses.

FIG. 14 is a diagram showing the insertion of an m-tert gene at a specific locus in a bacterial artificial chromosome in a mouse cytomegalovirus (MCMV). A M107 locus 1410 in a MCMV is bordered by a homologous recombination site on each side 1420, 1430, which are bordered by diagnostic primers 1440, 1450 respectively, allowing for the recombination of a desired gene cassette into a chromosome. According to a preferred aspect, an m-tert gene cassette 1460 is recombined at the point of the M107 locus 1410 in an MCMV BAC, resulting in the gene cassette 1460 now being opposed by homologous recombination sites 1420, 1430 and diagnostic primers 1440, 1450, at a specific point denoted by the M107 locus 1410 in the bacterial chromosome.

The skilled person will be aware of a range of possible modifications of the various embodiments described above. Accordingly, the present invention is defined by the claims and their equivalents.

1. A method for gene therapy using intranasal administration of genetically modified viral vectors, comprising:
selecting a plurality of target genes for therapeutic administration to a human patient;
creating a gene cassette comprising the plurality of target genes with terminal recombination sites;
inserting the gene cassette into a circular plasmid;
transfecting the circular plasmid into a bacterial cell;
sequencing DNA from the bacterial cell to ensure that the plurality of target genes is present in the bacterial DNA;
performing viral transduction to infect a human-administrable virus with the plurality of target genes in the bacterial cell;
preparing a solution containing a therapeutic amount of viral agents comprising the plurality of target genes; and
intranasally administering the solution into the human patient.

2. The method of claim 1, wherein the virus administered intranasally is a human cytomegalovirus.

3. The method of claim 1, wherein two or more target genes are administered to the human patient intranasally.

4. The method of claim 1, wherein the virus administered intranasally is a varicella zoster virus.

5. The method of claim 1, wherein a mouse cytomegalovirus is used to grow desired plasmids before transfection into a human cytomegalovirus.

6. The method of claim 3, wherein the virus administered intranasally is a human cytomegalovirus.

7. The method of claim 3, wherein the virus administered intranasally is a varicella zoster virus.
